(19) United States
(12) Patent Application Publication
(10) Pub. No.: US 2019/0209798 A1
(43) Pub. Date: Jul. 11, 2019

(54) Nasal Cannula Cover

(71) Applicant: Keisha LaShuna Landrum, Denver, CO (US)

(72) Inventor: Keisha LaShuna Landrum, Denver, CO (US)

(21) Appl. No.: 15/784,126

(22) Filed: Jan. 7, 2018

(51) Int. Cl. A61M 16/06; A61M 16/00

(52) U.S. Cl. CPC A61M 16/0666; A61M 2210/0618; A61M 2202/0208; A61M 16/0087

(57) ABSTRACT

The Nasal Cannula Cover will be utilized as an aid in infection prevention. The Nasal Cannula will be encased within the Nasal Cannula Cover. The Nasal Cannula Cover aids in the prevention of many foreign objects from being directly transmitted to the nasal cavity byway of the Nasal Cannula. In no way does the Nasal Cannula Cover replace existing sanitization or maintenance methods used for care of the Nasal Cannula. A Nasal Cannula Cover should be an accessory to every Nasal Cannula used or sold as an aid in infection prevention

Provisional Application cross-reference 62/408,761

### BACKGROUND OF CANNULA COVER

The Nasal Cannula Cover will be utilized as an aid in infection prevention. The Nasal Cannula has 2 small prong like tubes that are placed in the nostrils which allows oxygen flow into the nasal cavities. When the nasal cannula is not being used, the Nasal Cannula Cover will aid in protecting and preventing many foreign objects from touching, landing or resting on the Nasal Cannula by encasing it within the Nasal Cannula Cover. The Nasal Cannula Cover aids in the prevention of many foreign objects from being directly transmitted to the nasal cavity byway of the Nasal Cannula. In no way does the Nasal Cannula Cover replace existing sanitization or maintenance methods used for care of the Nasal Cannula.

Statistics show the global oxygen therapy market was USD 7 billion in 2015 and is expected to reach USD 18 billion by 2020. The oxygen therapy market end user was approximately 600 million and is anticipated to increase to 800 million by 2024 Respiratory diseases such as COPD, sleep apnea and asthma are driving the oxygen therapy market Therefore, there is no other product on the market and there is a global need for the Nasal Cannula Cover.

### BRIEF SUMMARY OF INVENTION

There are millions of users of the Nasal Cannula and each day have to find a way to protect the Nasal Cannula from elements of everyday life. The Nasal Cannula Cover that is lightweight can aid Nasal Cannula users in protecting their Nasal Cannula's.

### BRIEF DESCRIPTION OF DRAWINGS

FIG. 1. The Cannula Cover is shown open allowing you to see the length measurements.

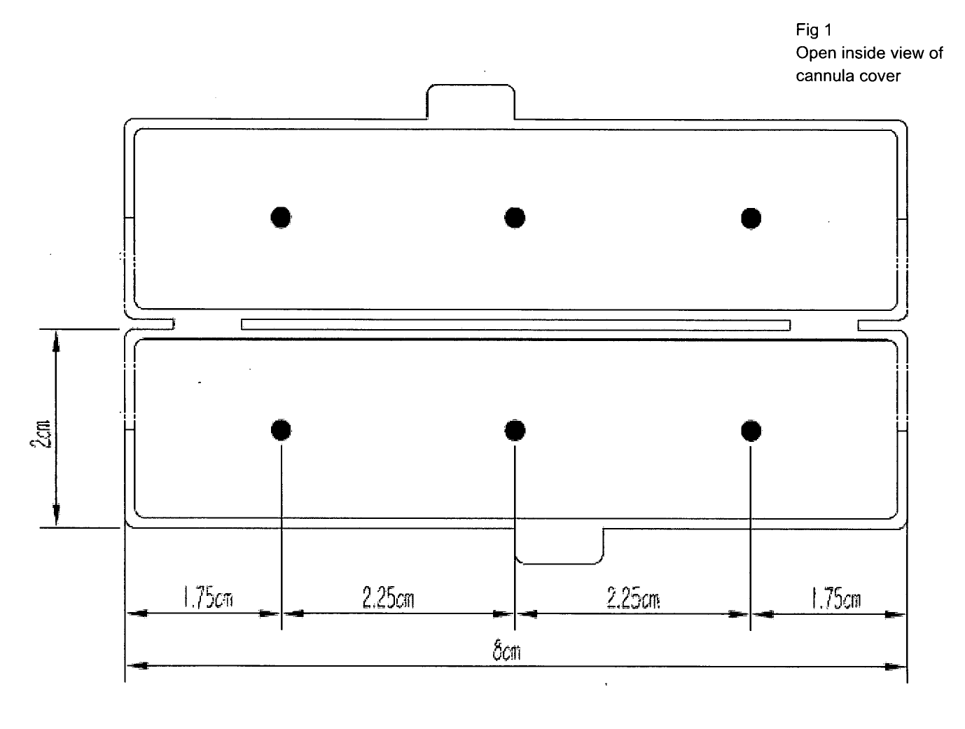

FIG. 2. The Cannula Cover is shown open allowing you to see the width measurements.

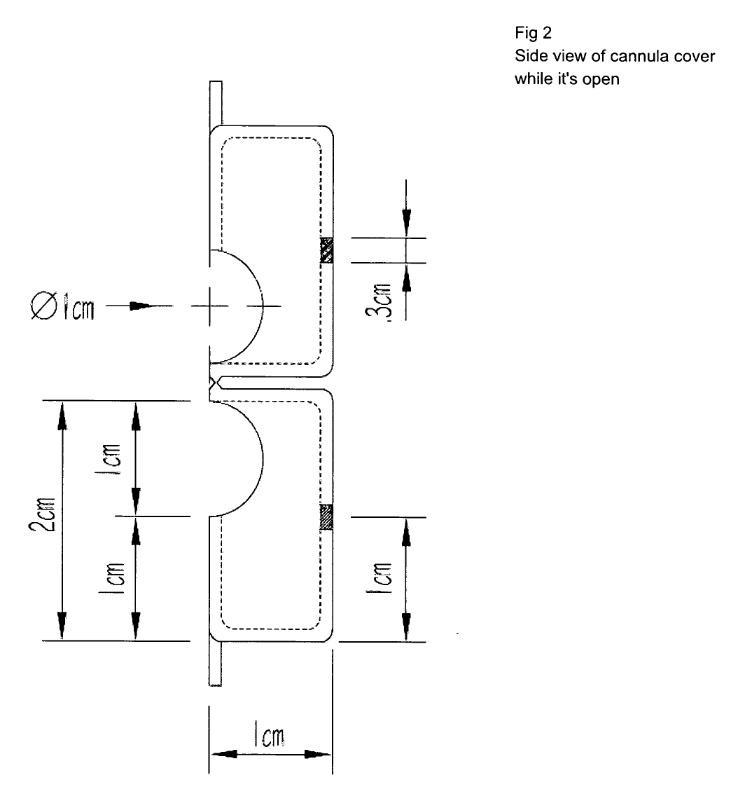

FIG. 3. The Cannula Cover is showing inside view with air vents exhaust.

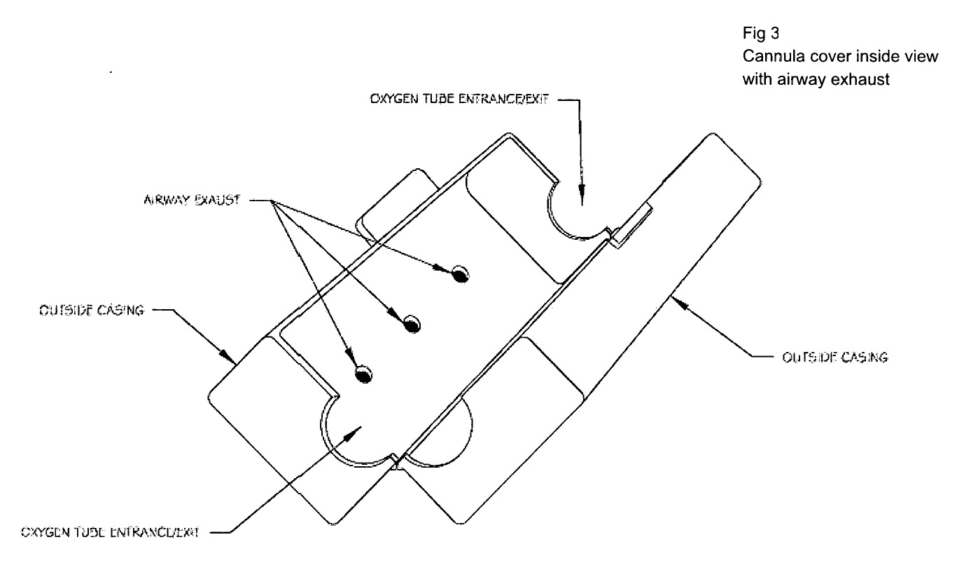

FIG. 4. The Cannula Cover is shown closed with a kiss closure and airway openings.

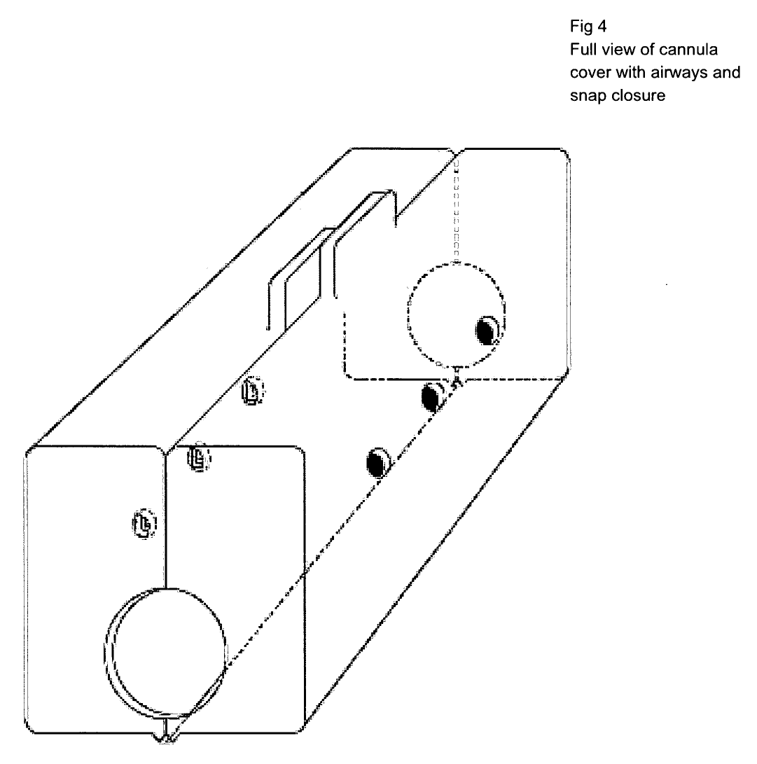

### DETAILED DESCRIPTION

The Nasal Cannula Cover is a one piece small rectangle shaped apparatus, that opens and closes with a kiss closure, a two-piece plastic hinged piece that has 3 holes for ventilation and an entrance and exit opening for the nasal cannula tubing. The Nasal Cannula Cover Measurements: 8.0 cm (L), 1.3 cm (H), 2.0 cm (W), 1.5 cm Diameter hole for (tubing entrance and exit) Construction: Plastic

1. An apparatus for covering the Nasal Cannula, to aid in the infection prevention process.
